(19) United States
(12) Patent Application Publication
(10) Pub. No.: US 2008/0218180 A1
(43) Pub. Date: Sep. 11, 2008

(54) Apparatus, a System and a Method for Enabling an Impedance Measurement

(75) Inventors: Eberhard Waffenschmidt, Aachen (DE); Claudia Igney, Karlsruhe (DE); Andreas Brauers, Aachen (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 11/995,427

(22) PCT Filed: Jun. 28, 2006
(86) PCT No.: PCT/IB06/52153

§ 371 Date: Jan. 11, 2008

(30) Foreign Application Priority Data
Jul. 13, 2005 (EP) 05106402.0

(51) Int. Cl. G01R 27/04; A61B 5/053

(52) U.S. Cl. 324/633; 600/547

(57) ABSTRACT

The invention relates to an apparatus (1) for impedance measurement of an external substance, said apparatus comprising a plurality of resonant circuits with respective coil elements (3a, 3b, 3c, 3d) and respective capacitive elements (5a, 5b, 5c, 5d), said resonant circuits operating at different resonant frequencies. The signals (S1, S2, S3, S4) from the resonant circuits are detected by an ampere meter (6). The power loss experienced by the resonant circuits due to an electromagnetic interaction with a conductive body is reflected in a change in the magnitude of respective signals. By detecting the signal (S1, S2, S3 or S4), the power loss by the resonant circuit is determined. The resonant circuit is preferably integrated into an insulating fabric carrier (2). The invention further relates to a vital sign measurement system and a method of enabling an impedance measurement.

The invention relates to an electromagnetic impedance measurement apparatus comprising a sensor element for enabling an impedance measurement of an external substance.

The invention further relates to a vital sign measurement system arranged to measure a signal representative of a vital sign of an individual.

The invention still further relates to a method of enabling an impedance measurement of an external substance.

An embodiment of the apparatus as set forth in the opening paragraph is known from US 2003/0055358 A1. The known apparatus is arranged to enable an electromagnetic bioimpedance measurement in biological tissue. For this purpose, the known apparatus comprises a single sensor element arranged to detect a signal representative of electrical eddy currents propagating in the tissue in response to an externally applied alternating magnetic field. The known apparatus is capable of determining the bioimpedance of a body segment corresponding to a cross-section of the sensor element.

It is a disadvantage of the known apparatus that in order to obtain a map of the bioimpedance values, the sensor element has to be displaced with respect to the surface of the tissue. This procedure is time consuming and may comprise substantial inaccuracies due to displacement errors.

It is an object of the invention to provide an apparatus for impedance measurements, whereby accurate spatially resolved measurement is enabled.

To this end, the apparatus according to the invention comprises a further sensor element for enabling a spatially resolved impedance measurement of said substance, said sensor element and said further sensor element being arranged as parts of respective resonant circuits operating at different resonant frequencies.

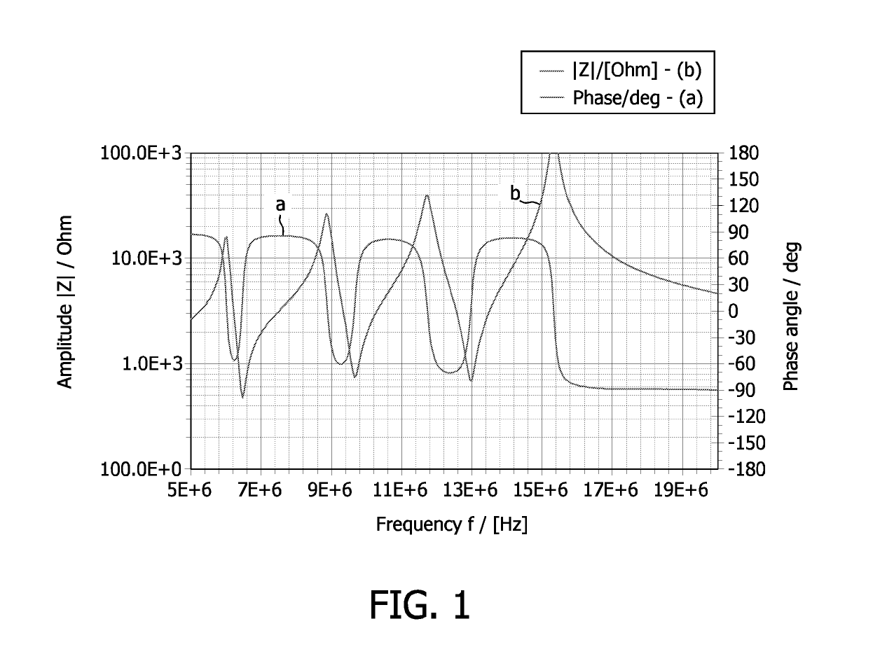

The technical measure according to the invention is based on the insight that arranging a plurality of sensor elements, for example two or more, in the vicinity of each other allows a spatially resolved impedance measurement. In order to enable an independent read-out of these sensor elements, each sensor is arranged as a part of a respective resonant circuit, with each resonant circuit being set to a different resonant frequency. Preferably, the difference between respective resonance frequencies is in the order of 10%. This principle has been experimentally validated, the results thereof being discussed with reference to FIG. 1. Thus, in accordance with the apparatus according to the invention, a measurement set-up is provided enabling a reliable impedance measurement over an area of interest with a spatial resolution. This fact is of particular importance for medical applications, where as a non-invasive, position-dependent measurement of such vital signs as breath action and depth, heart rate, change of heart volume, blood glucose level, etc. is enabled with a high accuracy of spatial resolution.

In an embodiment of the apparatus according to the invention, the sensor element and the further sensor element are conceived to form an array or a matrix of sensor elements.

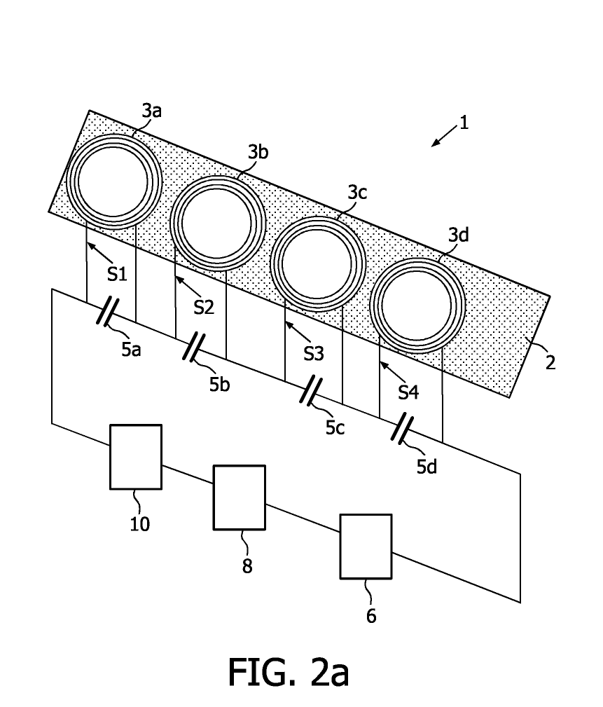

It is found to be particularly advantageous to design the sensor elements in the form of an array or matrix. The desired spatial resolution can be reached by appropriately choosing respective sizes of sensor elements. This particular embodiment will be discussed in more detail with reference to FIG. 2a and FIG. 2b.

In a further embodiment according to the invention, the sensor element and the further sensor element comprise respective sensor coils cooperating with respective capacitive elements, the respective resonant frequencies being determined by pre-selected values of the respective capacitive elements.

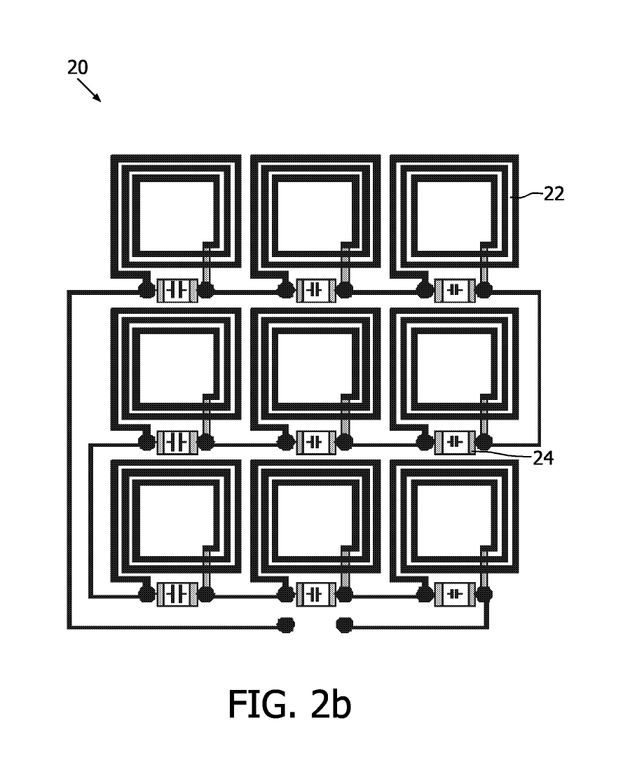

It is found to be particularly advantageous to use a combination of standard coil elements having pre-selected lengths of their connection tracks and a variety of capacitive elements, thus enabling the design of a plurality of resonant circuits having different respective resonant frequencies. Preferably, per se known Surface Mount Device (SMD) capacitors are used for capacitive elements. This arrangement is discussed in further detail with reference to FIG. 3. It is noted that, alternatively, it is possible to select standard capacitive elements with pre-selected values and to vary the resonant frequencies of the resonant circuits by changing the respective length of the connection tracks of the inductive coils.

In a still further embodiment of the apparatus according to the invention, the sensor element and the further sensor element are arranged in an immobilizing unit.

In some applications of the apparatus according to the invention, it may be desirable to enable an impedance measurement of an individual in circumstances where said individual is being positioned in a suitable immobilizing unit, for instance a chair, a bed, or the like. It must be noted that the apparatus according to the invention may just as well be used when the individual carries out a task while being positioned in the immobilizing unit. For example, such a task may be operating a vehicle, carrying out stationary labor when sitting in an office, or the like. In these examples the apparatus according to the invention is suitable for performing an isolated impedance measurement, or for monitoring any change in a series of impedance measurements.

In a still further embodiment of the apparatus according to the invention, the sensor element and the further sensor element are arranged in a wearable piece.

It is found particularly advantageous to arrange the apparatus according to the invention in a wearable piece, like a T-shirt, an underwear piece, armbands, or the like. This embodiment is particularly advantageous for enabling repetitive impedance measurements of moving individuals, for example for sport coaching or monitoring rehabilitating patients.

The vital sign measurement system according to the invention comprises the apparatus as discussed with reference to the foregoing.

The measurement of the bioimpedance is used to measure various vital parameters of a human body, preferably in a contactless way. By incorporating the apparatus according to the invention into the vital sign measurement system, an alternating magnetic field is induced in a part of the human body. This alternating magnetic field causes eddy currents in the tissue of the body. Depending on the type of tissue these eddy currents are stronger or weaker. The eddy currents cause losses in the tissue, which can be measured, for example, as a decrease of the quality factor of the inductor loop. They also cause a secondary magnetic field, which can be measured as an inductivity change of the inductor loop or, alternatively, as an induced voltage in a second inductor loop. By using a plurality of sensor elements operating at different resonant frequencies, it is possible to provide a measurement system capable of providing a spatially resolved measurement of such vital signs as breath action and depth, heart rate, change in heart volume, blood glucose level, fat or water content of a selected tissue, lung edema and edema in peripherals, etc.

The method according to the invention comprises the steps of:
providing an apparatus comprising a sensor element and a further sensor element for enabling a spatially resolved impedance measurement of said substance, said sensor element and said further sensor element being arranged as parts of respective resonant circuits operating at different resonant frequencies;
positioning the apparatus in the vicinity of the substance;
applying alternating electromagnetic fields to the sensor element and the further sensor element;
detecting a signal representative of a variation of respective quality factors of said resonance circuits.

The method according to the invention is particularly suitable for performing mapping of a certain vital sign, which can be detected by means of spatially resolved bioimpedance measurement.

These and other aspects of the invention will be discussed with reference to Figures.

FIG. 1 presents, in a schematic way, results of a measured impedance spectrum of a series-connected sensor array.

FIG. 2a presents, in a schematic way, an embodiment of a sensor array according to the invention.

FIG. 2b presents, in a schematic way, an embodiment of a matrix array of sensor elements according to the invention.

FIG. 3 presents, in a schematic way, an embodiment of the apparatus according to the invention, where resonant circuits are designed using SMD capacitors.

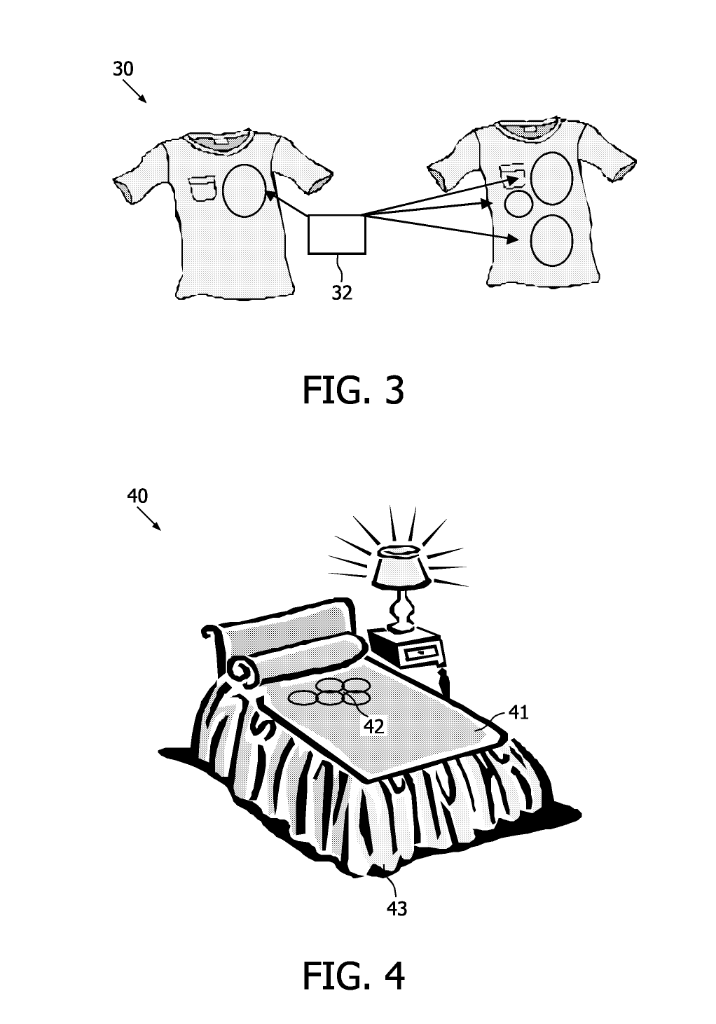

FIG. 4 presents schematically an embodiment of a system for monitoring according to the invention, where the magnetic means are integrated into clothing.

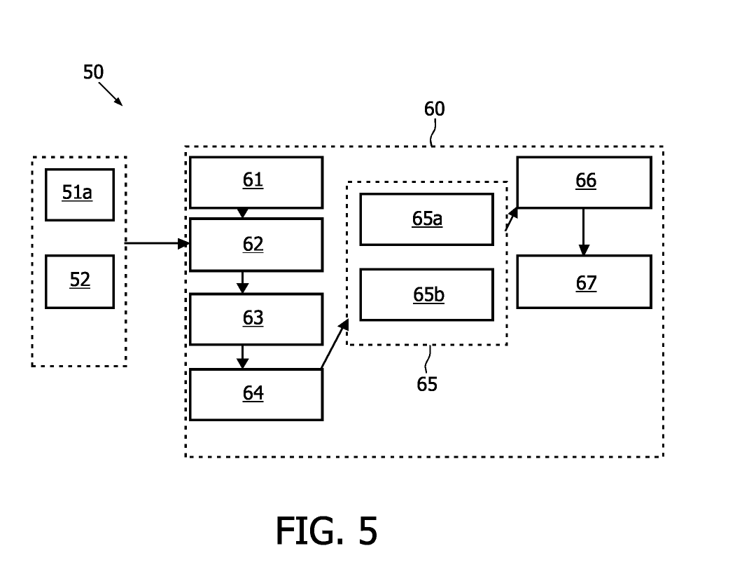

FIG. 5 presents schematically an embodiment of a system for monitoring according to the invention, said system comprising further sensing means.

FIG. 1 presents, in a schematic way, results of a measured impedance spectrum of a series-connected sensor array. This Figure shows a phase (curve a) and an amplitude (curve b) as respective functions of an external RF-field, curve b being presented in logarithmic scale. In this example, the sensor array comprises spiral copper tracks arranged on a Polyimide (“Flexfoil”) substrate. They constitute four respective resonant circuits with different resonant frequencies corresponding to four sensor elements. In general, to describe a frequency difference between the respective resonant frequencies, the quality factor of the resonance peaks can be used. As a rule, the lower the quality factor, the wider the peaks are and the larger the selected distance between them. Preferably, the frequency difference is selected at a value of at least 3 times the df-value (describing the width of the peak at −3 dB). This df-value results from dividing the resonant frequency fᵣₑₛ by the quality factor Q; it is df=fᵣₑₛ/Q. For typical applications this will result in a frequency difference in the order of about 10%. The resonant circuits are constructed by selecting coils with different lengths of their connection tracks. The impedance spectrum shown in FIG. 1 shows the voltage across the array, measured with a constant current. FIG. 1 shows clearly four resonant peaks (curve “b”) corresponding to each of the four resonant circuits. Curve “a” in FIG. 1 shows corresponding phase data measurements. It is experimentally established that only one of the resonances, i.e. the one related to a certain coil, substantially changes its quality factor and the resonant frequency, if conducting matter is placed close to it. Neighboring resonance peaks are affected to a much smaller extent. Thus, it is demonstrated that the frequency can be used to achieve spatial resolution of an impedance measurement.

FIG. 2a presents, in a schematic way, an embodiment of a sensor array according to the invention. FIG. 2 presents schematically an embodiment of the apparatus 1 according to the invention, comprising a plurality of resonant circuits having respective coil elements 3a, 3b, 3c, 3d and respective capacitive elements 5a, 5b, 5c, 5d. Power supply means 8 energize the resonant circuits so that oscillating magnetic fields (not shown) are produced. The signals S1, S2, S3, S4 from the resonant circuits are detected by an ampere meter 6. The power loss experienced by the resonant circuits due to an electromagnetic interaction with a conductive body (not shown) is reflected in a change in the magnitude of respective signals. By detecting the signal S1, S2, S3 or S4, the power loss by the resonant circuit is determined. In case the relation between the absolute value of the power loss and the signal S is known, the conductive characteristics of the volume being investigated can be determined. In order to ensure a constant power load, the resonant circuit preferably is enabled with a feedback loop 10. The feedback loop is preferably arranged so that the voltage controlling the amplitude of the resonant circuit is proportional to the RF power delivered by the resonant circuit. The resonant circuit is preferably integrated into an insulating fabric carrier 2. Preferably, the conductors forming the coils 3a, 3b, 3c, 3d are interwoven with threads of fabric 2. Still preferably, the sensor element and the further sensor element comprise flexible material. An example of a suitable flexible material is a Polyimide (“Flexfoil”) substrate. It is noted that a variety of possible embodiments of a flexible material are envisaged; therefore, the present example should not be construed as limiting the scope of the invention. The advantage of the array arrangement is that it can easily be extended.

FIG. 2b presents, in a schematic way, an embodiment of a matrix array of sensor elements according to the invention. Although in this particular example the matrix 20 comprises a square arrangement, it is also possible to have an X by Y matrix arrangement, or any irregular arrangement of sensor elements 22. Preferably, the sensor elements, comprising coils of type 22, are connected to SMD (surface Mount Device) parallel capacitors 24. Preferably, each of the SMD capacitors 24 is slightly different, which is indicated by varying the size of the respective symbols in FIG. 2b. As noted earlier, the different resonant frequencies can be achieved by using different connection tracks for the coils 22 as a single measure, or in addition to variable SMD capacitors.

FIG. 3 presents schematically an embodiment of a system of the apparatus 40 according to the invention, where the sensor means are integrated into clothing. In a simplest embodiment, a T-shirt is used as an insulating fabric carrier to be integrated with resonant circuits 32. Here the resonant circuit 32 comprises all units discussed with reference to FIG. 1. In an alternative embodiment, the designed measurement system can comprise a plurality of arrangements for impedance measurement.

FIG. 4 presents schematically a further embodiment of the apparatus according to the invention, said apparatus comprising an immobilizing unit 41 whereon the sensing means 42 are mounted. In this embodiment, a bed 41 is used to accommodate a person (not shown). A bed sheet 43 is provided with a plurality of sensing means 42, as discussed with reference to FIG. 2a.

FIG. 5 presents schematically an embodiment of the vital sign measurement system according to the invention. The vital sign measurement system 50 comprises sensor means 51 arranged to monitor a physiological condition of the user by carrying out an impedance measurement as discussed with reference to FIG. 2a. The sensor means 51 comprises a suitable plurality of resonant circuits 51a to be arranged in the vicinity of the body of a user to pick-up a signal characteristic of the targeted physiological condition, for example a signal related to breath action, breath depth, heart rate, a change of heart volume, blood glucose level, fat or water content of a tissue, like lung edema, edema in peripherals, and the like. Additionally, the sensor means 51 can comprise a further sensor means 52 arranged to monitor a reference signal, for example, from a healthy tissue of the same user. The sensor means 51 is preferably arranged to perform continuous monitoring of the physiological condition of the user and is further arranged to provide a corresponding signal to the front-end electronics 60 of the system 50. The sensor means 51 and the front-end electronics 60 are worn on the body of the user, preferably at the thorax area. Alternatively, the sensor means 51 can be integrated into a piece of furniture, a bed sheet, a safety belt, a vehicle seat, etc. Examples of suitable fabric carriers for the wearable device are known per se in the art. The front-end electronics 60 is arranged to analyze the signal from the resonant circuit 51a. For that purpose, the front-end electronics 60 comprises a preamplifier 61 and an analogue processing circuit 62, an ADC unit 63, detection means 65 and a μ-processor 64. The front-end electronics 60 may further comprise suitable alarm means 66 and transmission means 67. A signal detection means 65 comprises a sensor signal interpretation unit 65a and feature extraction means 65b. The system 60 operates as follows: the sensor means 51 acquires the raw data, which are delivered to the front-end electronics 60. The front-end electronics 60 provides means for receiving the signals from the sensor means, performs suited analogue processing by means of the analogue processing circuit 62. The processed raw data are converted into a digital format by means of the ADC 63 and are forwarded by a μ-processor 64 to the detection means 65, where the condition of the user is analyzed. The detection means 65 comprise a sensor signal interpretation unit 65a arranged to derive a feature in the signal characteristic, for example a feature indicative of an abnormal physiological condition of the user. For cardiac applications, for example said feature can be the amplitude of the signal. In case the detection means 65 detects the abnormal condition, a signal is sent to the alarm means 66 to generate an alarm, which is transmitted by the transmitting means 67, for example by means of a RF-link, to warn a user, or a bystander, or specialized medical personnel.

1. An electromagnetic impedance measurement apparatus comprising a sensor element for enabling an impedance measurement of an external substance, said apparatus further comprising a further sensor element for enabling a spatially resolved impedance measurement of said substance, said sensor element and said further sensor element being arranged as parts of respective resonant circuits operating at different resonant frequencies.

2. An apparatus according to claim 1, wherein the sensor element and the further sensor element are conceived to form an array or a matrix of sensor elements.

3. An apparatus according to claim 1, wherein the sensor element and the further sensor element comprise respective sensor coils cooperating with respective capacitive elements, the respective resonant frequencies being determined by pre-selected values of the respective capacitive elements.

4. An apparatus according to claim 1, wherein the sensor element and the further sensor element comprise respective sensor coils cooperating with respective capacitive elements, the respective resonant frequencies being determined by respective lengths of connection tracks of the sensor element and the further sensor element.

5. An apparatus according to claim 1, wherein the sensor element and the further sensor element are arranged in an immobilizing unit.

6. An apparatus according to claim 1, wherein the sensor element and the further sensor element are arranged in a wearable piece.

7. An apparatus according to claim 1, wherein the sensor element and the further sensor element comprise flexible material.

8. A vital sign measurement system arranged to measure a signal representative of a vital sign of an individual, said system comprising the apparatus according to claim 1.

9. A method of enabling an impedance measurement of an external substance, said method comprising the steps of:
providing an apparatus comprising a sensor element and a further sensor element for enabling a spatially resolved impedance measurement of said substance, said sensor element and said further sensor element being arranged as parts of respective resonant circuits operating at different resonant frequencies;
positioning the apparatus in the vicinity of the substance;
applying alternating electromagnetic fields to the sensor element and the further sensor element;
detecting a signal representative of a variation of respective quality lectors of said resonance circuits.

10. A vital sign measurement device comprising:
two or more sensor elements positioned in the vicinity of each other; wherein the two or more sensors have different resonant frequencies, such that the two or more sensors have an independent read-out when an alternating electromagnetic field is applied;
wherein the two or more sensors provide signals used to determine a vital sign.

11. The vital sign measurement device of claim 10 wherein the resonant frequencies of the two or more sensors differ by approximately ten percent.

12. The vital sign measurement device of claim 10 further comprising a wearable piece in which the two or more sensors are positioned.

13. The vital sign measurement device of claim 10 further comprising an immobilizing unit in which the two or more sensors are positioned.

14. The vital sign measurement device of claim 10 wherein the two or more sensors are sized to provide a desired spatial resolution.
